(19) United States
(12) Patent Application Publication
(10) Pub. No.: US 2011/0152924 A1
(43) Pub. Date: Jun. 23, 2011

(54) OXIDIZED REGENERATED CELLULOSE ADHESIVE TAPE

(76) Inventors: Michel Gensini, Flemington, NJ (US); Sanda Gonzalez, Bedminster, NJ (US)

(21) Appl. No.: 12/645,164

(22) Filed: Dec. 22, 2009

(51) Int. Cl. A61B 17/03; B05D 3/10

(52) U.S. Cl. 606/213; 427/2.31

(57) ABSTRACT

A novel ORC adhesive tape is disclosed. Specifically, the ORC adhesive tape is useful as an alternative to conventional mechanical and non-mechanical wound closure means. The ORC tape has a tape substrate made from neutralized ORC and it contains an adhesive composition such as a bioabsorbable alpha-cyanoacrylate.

### FIELD OF THE INVENTION

The field of art to which this invention relates is wound closure, more specifically, medical devices for wound closure.

### BACKGROUND OF THE INVENTION

Conventional methods of wound closure include sutures and staples to provide adequate wound support and tissue approximation for the duration of wound healing. In both techniques the application of the wound closure devices involves additional trauma to the wound in terms of creating tissue pathways for receiving the devices. In addition, when either of these methods is used to close wounds inside the body, especially when sealing or attaching organs containing fluids, for example, intestine, blood vessels, and lungs, there is potential for fluid leaks that may cause complications and higher morbidity rates.

Direct application of biocompatible adhesives have also been proposed and used for wound closure, especially alpha-cyanoacrylates, but their use is somewhat limited by the biodegradability and biocompatibility of existing compositions. It is generally known that monomers of alpha-cyanoacrylates are extremely reactive, polymerizing rapidly in the presence of minute amounts of an initiator including moisture present in the air or on moist surfaces such as tissue. The combination of α cyanoacrylates with a flexible material has been described in US Patent applications 20080255610 (A1), 20090076542(A1), and 20050182443 (A1), and are particularly useful for topical applications, where biodegradability of the flexible material is not necessary since it can be removed later after wound healing. However, the combination of α cyanoacrylate adhesive and an absorbable material such as oxidized regenerated cellulose has not been described.

Oxidized regenerated cellulose (ORC) is well known in the medical field as hemostatic agent or adhesion prevention material. It presents self-adherence when hydrating from moisture from surrounding tissues. However, ORC has never been used to approximate two sections of tissue together because it lacks mechanical strength. ORC is also acidic in nature due to the pendant carboxylic acid groups. The combination of α cyanoacrylates with oxidized regenerated cellulose has not been considered before because the polymerization rate of cyanoacrylate molecules is acid sensitive. Thus, the ORC surface pH naturally will prevent polymerization of the cyanoacrylate.

Accordingly, there is a need in this art for novel tissue approximation devices incorporating tissue adhesives

### SUMMARY OF THE INVENTION

An ORC adhesive tape device for wound closure is disclosed. The device has a partially neutralized, lyophilized ORC structure or substrate combined with an alpha-cyanoacrylate monomer which polymerizes and attaches the ORC to the wound site.

Another aspect of the present invention is a method of approximating or treating tissue using the above-described device.

Yet another aspect of the present invention is a kit containing the above-described device wherein the ORC structure or substrate is maintained separately from the alpha-cyanoacrylate monomer, and combined in the field prior to use by the surgeon or healthcare provider.

These and other aspects and advantages of the present invention will become more apparent from the following description and accompanying drawings.

### BRIEF DESCRIPTION OF THE FIGURES

FIG. 1 is a graph illustrating burst strength at wound failure for four treatment groups: ORC alone, cyanoacrylate alone, ORC adhesive tape on dry tissue, ORC adhesive tape on wet tissue.

FIG. 2 is a graph illustrating burst strength at wound failure for three treatment groups: cyanoacrylate alone, ORC adhesive tape on dry tissue where ORC was dried on paper, ORC adhesive tape on dry tissue where ORC was lyophilized.

FIG. 3 is a schematic of a process for manufacturing the novel adhesive tapes of the present invention.

### DETAILED DESCRIPTION OF THE INVENTION

The present invention provides the combination of an oxidized regenerated cellulose (ORC) adhesive tape substrate wherein the substrate is a partially neutralized ORC fabric and an alpha-cyanoacrylate adhesive composition.

Partially neutralized ORC refers to ORC that has been modified from its original state by neutralizing some of its carboxylic functionality such that the pH in water of the resulting substrate is higher than the pH in water of the ORC starting material.

ORC is provided in various forms such as woven fabrics, nonwoven fabrics, foams, particles, and the like. In one embodiment, the ORC is a nonwoven fabric such as ORC sold under the tradename SURGICEL FIBRILLAR, by Ethicon, Inc. in Somerville, N.J. In another embodiment, the ORC is a woven fabric such as those sold under the tradenames INTERCEED, SURGICEL, and SURGICEL NU-KNIT by Ethicon, Inc. in Somerville, N.J. The ORC may be used by itself or in combination with other absorbable materials, such as absorbable polyester materials or other polysaccharides. The ORC may be in any size or shape suitable for closing a wound. Suitable shapes include, but are not limited to square, rectangular, oval, triangular, polygonal, circular, semi-circular and the like.

The ORC useful in the tape devices of the present invention is partially neutralized such that the acidic nature of the surface will not interfere with the anionic polymerization of the cyanoacrylate while in contact with moist tissue. ORC is neutralized by treating the oxidized cellulose with a water solution or alcohol solution of a basic salt of a weak organic acid. The ORC also needs to be completely dried prior to combining with the cyanoacrylate such that the cyanoacrylate will not polymerize prematurely.

The ORC is partially neutralized in an amount or to a degree sufficiently effective to allow the alpha-cyanoacrylate to polymerize when implanted in a mammalian subject. The amount (i.e., the degree or extent) of neutralization necessary to allow the alpha-cyanoacrylate to polymerize is dependent upon the particular alpha-cyanoacrylate in use. In one embodiment, the ORC is neutralized to a pH of from about 2 to about 9. In another embodiment, the ORC is neutralized to a pH of from about 5 to about 7.

ORC is partially neutralized by treating the ORC with an aqueous solution or alcohol solution of a basic salt of a weak acid, a weak base, or a dilute solution of a strong base. Suitable alcohols include, but are not limited to methanol, ethanol, isopropanol and the like. Suitable basic salts (i.e. sodium, potassium, magnesium, etc.) of weak acids are organic or inorganic weak acids including, but not limited to bicarbonates, acetates, and the like. Suitable bases include, but are not limited to hydroxides, amines, and the like. In one embodiment, the aqueous solution is a water solution of sodium bicarbonate. The concentration of the basic salt of a weak acid, a weak base, or a dilute solution of a strong base in the aqueous or alcohol solution will depend upon the strength of the acid or base used. For example, in the case of sodium bicarbonate a suitable concentration is about 1% by weight.

As mentioned above, the ORC is partially neutralized by immersing the ORC in the aqueous solution or alcohol solution of a basic salt of a weak acid, a weak base, or a dilute solution of a strong base a temperature of about 32° C. (room temperature) or cooler. The ORC is immersed in the solution for a sufficiently effective period of time to partially neutralize the ORC. The amount of time that the ORC resides in the solution is dependent upon a number of factors including, the strength and concentration of the solution, degree of oxidation of the ORC, how tight the weave or density of the fabric, and the like. One of skilled in the art will be able to weigh these factors and determine the appropriate amount of time for immersion in the solution given this disclosure.

In addition to partially neutralizing the ORC, the partially neutralized ORC is dried to preserve the neutralized ORC from re-acidification and thus increase its useful shelf life. ORC may be dried by conventional methods commonly known in the art such as, pat or blot dry with paper, vacuum drying, and lyophilization. However it is particularly preferred to utilize both patting and blotting the ORC dry after neutralization followed by lyophilzation to provide a superior result in obtaining a substantially dry ORC after neutralization. Lyophilization dries the ORC and removes enough residual moisture so that neutralized ORC can be stored dry (i.e., moisture-dry) and packaged with the cyanoacrylate as a one-component kit without premature polymerization of the cyanoacrylate.

The alpha-cyanoacrylate is preferably a bioabsorbable cyanoacrylate such as those described in U.S. Pat. No. 7,238,828B2. Specifically, the alpha-cyanoacrylate monomer is an alkyl ester alpha-cyanoacrylate monomer of the general formula having a spacer R1:

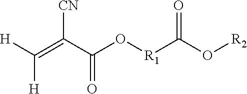

Wherein

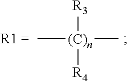

n is from 2 to 12; R3 and R4 are each an alkyl group or a hydrogen, and at least one of R3 or R4 is an alkyl group (e.g. linear or branched, or cyclic) having from 1, 2, 3, 4, 5, 6, 7, 8, 9, 10, 11, 12 and 13 carbon atoms; R2 is an alkyl group (e.g. linear or branched, or cyclic) having from 1, 2, 3, 4, 5, 6, 7, 8, 9, 10, 11, 12 and 13 carbon atoms; and the combined number of carbon atoms (N) in the spacer R1 is at least n+1.

It is important that the value of n is in a range such that it allows for feasible preparation and purification of the composition while providing desirable biodegradability and adhesive properties. A preferred range for n is from about 2 to 12, a more preferred range is from about 2 to 8.

The combined number of carbon atoms (N) is defined as the combined value of the number of carbon atoms on the R3 and R4 side branches and the number of carbon atoms on the spacer backbone (n), wherein the combined number of carbon atoms (N) in

is at least n+1.

A preferred combined number of carbon atoms (N) in

is 4 or greater.

A more preferred combined number of carbon atoms (N) in

is 5 or greater.

Examples of the monomers include, but are not limited to alkyl ester alpha-cyanoacrylate monomer, such as 3-(2-cyano-acryloyloxy)-butyric acid ethyl ester, 3-(2-cyano-acryloyloxy)-pentanoic acid ethyl ester, 3-(2-cyano-acryloyloxy)-hexanoic acid ethyl ester, 3-(2-cyano-acryloyloxy)-heptanoic acid ethyl ester, 3-(2-cyano-acryloyloxy)-octanoic acid ethyl ester, 4-(2-cyano-acryloyloxy)-hexanoic acid ethyl ester, 5-(2-cyano-acryloyloxy)-hexanoic acid ethyl ester, ethyl lactoyl cyanoacrylate, n-propyl lactoyl cyanoacrylate, isopropyl lactoyl cyanoacrylate, n-butyl lactoyl cyanoacrylate, isobutyl lactoyl cyanoacrylate, pentyl lactoyl cyanoacrylate, hexyl lactoyl cyanoacrylate, 2-ethyl-hexyl-lactoyl cyanoacrylate, n-octyl lactoyl cyanoacrylate, iso-octyl lactoyl cyanoacrylate, ethyl glycoloyl cyanoacrylate, n-propyl glycoloyl cyanoacrylate, isopropyl glycoloyl cyanoacrylate, n-butyl glycoloyl cyanoacrylate, isobutyl glycoloyl cyanoacrylate, pentyl glycoloyl cyanoacrylate, hexyl glycoloyl cyanoacrylate, 2-ethyl-hexyl-glycoloyl cyanoacrylate, n-octyl glycoloyl cyanoacrylate, and iso-octyl glycoloyl cyanoacrylate. In one embodiment, the alkyl ester alpha-cyanoacrylate monomer is selected from the group consisting of 3-(2-cyano-acryloyloxy)-butyric acid ethyl ester, 3-(2-cyano-acryloyloxy)-pentanoic acid ethyl ester, 3-(2-cyano-acryloyloxy)-hexanoic acid ethyl ester, 3-(2-cyano-acryloyloxy)-heptanoic acid ethyl ester, 3-(2-cyano-acryloyloxy)-octanoic acid ethyl ester, 4-(2-cyano-acryloyloxy)-hexanoic acid ethyl ester, and 5-(2-cyano-acryloyloxy)-hexanoic acid ethyl ester. In another embodiment, the alkyl ester alpha-cyanoacrylate monomer is 3-(2-cyano-acryloyloxy)-hexanoic acid ethyl ester. The alkyl ester alpha-cyanoacrylate monomer may be employed individually or as a co-monomer with one or more alkyl ester alpha-cyanoacrylate monomer or other monomers such as alkyl cyanoacrylate and alkoxyalkyl cyanoacrylate including, but not limited to, methyl cyanoacrylate, ethyl cyanoacrylate, n-butyl cyanoacrylate, isobutyl cyanoacrylate, n-octyl cyanoacrylate, 2-octyl cyanoacrylate, dodecyl cyanoacrylate, hexyl cyanoacrylate, 2-ethylhexyl cyanoacrylate, methoxyethyl cyanoacrylate, 2-ethoxyethyl cyanoacrylate, 3-methoxybutyl cyanoacrylate, 2-butoxyethyl cyanoacrylate, 2-isopropoxyethyl cyanoacrylate, and 1-methoxy-2-propyl cyanoacrylate.

The ORC tape devices of the present invention are combined with the adhesive as follows. The adhesive may be applied to the ORC substrate in either a continuous or discontinuous manner. Thus, for example, the adhesive can be applied as a continuous layer over a desired area, or in a set or random pattern. Preferably, the adhesive is applied as a continuous layer on one or both sides of the ORC tape substrate, e.g., top or bottom surfaces. In this embodiment, the adhesive can be located on substantially the entire surface of the ORC. Alternatively, the adhesive coating is discontinuous to provide areas that are not covered by the adhesive, such as by the adhesive being provided in a form of regular or random spots, lines, or the like. In addition, in another embodiment, the entire substrate is saturated with the adhesive, including the top and bottom surfaces.

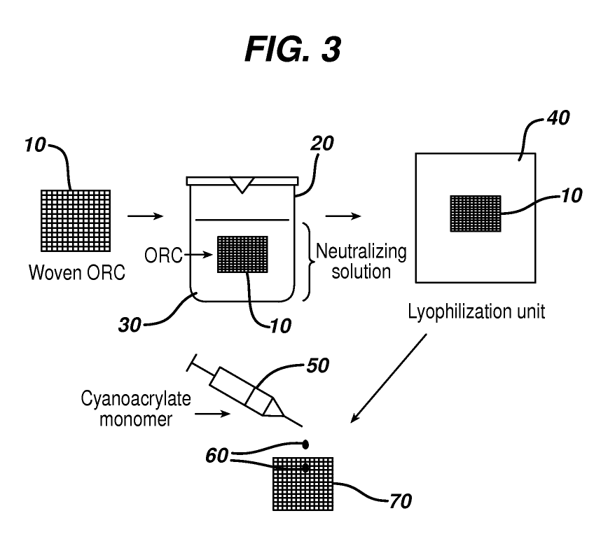

Referring to FIG. 3, a schematic illustrating a manufacturing process for the adhesive tapes of the present invention is illustrated. An ORC substrate 10 is immersed in vessel 20 containing neutralizing solution or bath 30. The neutralized ORC substrate 10 is removed from bath 30 and vessel 20 and further dired in lyophilization unit 40. If desired the wet substrate may be blotted to dry or partially it prior to drying in the lyophilization unit 40. Then an adhesive such as cyanoacrylic monomer 60 is applied to substrate 10 by syringe 50 to form device 10 of the present invention.

The adhesive tapes of the present invention are packaged in conventional packages that provide a sterile barrier. The devices of the present invention may be sterilized in the following manner: the ORC may be sterilized by conventional irradiation methods such as gamma, e-beam, and the like and separately the cyanoacrylate adhesive may be sterilized using dry heat. The cyanoacrylate may then be combined with the ORC by conventional aseptic preparation methods and packaged under vacuum or inert atmosphere. Optionally, a desiccant may be included in the packaging.

The alpha-cyanoacrylate adhesive is applied to the lyophilized neutralized ORC tape substrate prior to packaging, to provide a ready-to-use product that is pre-loaded to the ORC tape substrate. The pre-packaged product may be applied directly to the wound without further manipulation.

Alternatively, the alpha-cyanoacrylate adhesive may be applied to the ORC by the physician just prior to use, when the device is supplied in kit form. The ORC can be applied to the wound in much the same manner as a piece of tape, where substantially the entire surface of the ORC adheres to the wound. The cyanoacrylate adhesive composition can then be applied to the exposed surface of the ORC, in the manner as described above. A benefit of this embodiment is that the entire applied flexible substrate can be retained on the desired surface.

The adhesive is typically present in coat weight from about 10 to about 500, or preferably from about 50 to 150 micrograms per square inch. Other coat weights of the adhesive substance can be used, as desired. Preferably, the cyanoacrylate adhesive is the only attachment means present on the ORC for attaching the ORC to treatment site. Although not preferred, the ORC tape device of the present invention may incorporate other physical attachment means such as hooks, barbs, pins, projections, or the like, which operate to physically latch or otherwise attach the flexible substrate to the desired application or treatment site. However, it is preferred that the ORC tape device of the present invention not include features that penetrate the underlying tissue.

In addition to the dry, partially neutralized ORC and an alpha-cyanoacrylate adhesive composition, the ORC adhesive tape can, if desired, include one or more chemical materials located within the ORC or the adhesive composition, or both. For example, one or more chemical substances can be dispersed in the ORC or the adhesive composition, such as being chemically bound, physically bound, absorbed or adsorbed to the ORC. Thus, for example, the ORC can include a polymerization initiator or rate modifiers. The rate modifier can also be added to the adhesive composition but it has to be done immediately before the application of the adhesive composition to the ORC.

Both the ORC or the adhesive composition can include one or more materials such as chemicals like a plasticizing agent that assists in imparting flexibility to the polymer formed from the monomer. Examples of suitable plasticizers include but are not limited to tributyl citrate, acetyl tri-n-butyl citrate, polymethylmethacrylate, polydimethylsiloxane, hexadimethylsilazane and the like. The composition may also optionally include at least one thixotropic agent. Suitable thixotropic agents can include silica gels, such as those treated with a silyl isocyanate and optionally surface treated titanium dioxide.

The adhesive composition may also include thickeners. Suitable thickeners may include poly (2-ethylhexylmethacrylate), poly(2-ethylhexylacrylate) and others known to those skilled in the art.

The adhesive composition may optionally also include one or more stabilizers, preferably both at least one anionic vapor phase stabilizer and at least one anionic liquid phase stabilizer. These stabilizer agents may inhibit premature polymerization. Suitable stabilizers may include those listed in U.S. Pat. No. 6,183,593, the disclosure of which is incorporated by reference herein in its entirety.

The adhesive composition may include cross-linking agents in order to improve the cohesive strength of the polymer formed from the monomer. Such cross-linking agents are reported in U.S. Pat. No. 3,940,362 to Overhults, which is here by incorporated herein in its entirety by reference.

In addition, the ORC adhesive tape of this invention may further contain colorants such as dyes, pigments and pigment dyes. The colorant may be administered by being chemically bound, physically bound, absorbed or adsorbed to the ORC or dispersed within the adhesive composition.

The adhesive composition may also contain one or more preservatives, and methods for selecting them and incorporating then into adhesive compositions are disclosed in U.S. Pat. No. 6,579,469, the entire disclosure of which is incorporated herein by reference.

In embodiments of the present invention, the ORC adhesive tape may also optionally include at least one biological or therapeutical agent. In general, biological/therapeutical agents which may be administered by being chemically bound, physically bound, absorbed or adsorbed to the ORC or the adhesive composition include, without limitation, antiinfectives, such as antibiotics, antimicrobial agents, and antiviral agents; analgesics and analgesic combinations; antiinflamatory agents, immunosupressives; sedatives; tranquilizers; naturally derived or genetically engineered proteins; polysaccharides; procoagulants and hemostatic agents, such as prothrombine, thrombin, fibrinogen, fibrin, fibronectin, heparinease, etc.

The ORC tape device of the present invention is useful for hemostasis and wound closure, and with the appropriate amount of alpha-cyanoacrylate, adhesion prevention, with the ORC side opposed to the wound. For example, the ORC adhesive tape can be used as a replacement for conventional mechanical wound closure devices s such as sutures and staples and the like; and also for tissue sealants such as fibrin glues, gelatin-thrombin combinations, albumin-polyethylene products, polyethylene glycol hydrogels, etc. As compared to conventional wound closure means, the ORC adhesive tape device of the present invention generally provides the same wound approximation and closure strength benefits. The ORC adhesive tape provides significant benefits over the conventional wound closure means in terms of improved wound management, stronger adhesion to the underlying application site for non mechanical wound closure methods, improved patient satisfaction, and the like.

The ORC adhesive tape as described herein is useful as a wound closure device and may be used to approximate the edges of a wound. The ORC adhesive tape is particularly useful for closing internal wounds. The width of the device should extend at least a half inch beyond the wound edges. The length of the flexible substrate can be longer than the wound to be closed, and extend beyond opposite ends of the wound a sufficient distance to permit sufficient bonding. The edges of the wound are approximated and then the device is positioned on top and around the two edges of the approximated wound. The device is allowed enough time for the monomer to polymerize.

For example this device can be used as a standalone to approximate edges of a tubular organ e.g., gastrointestinal tract, blood vessels, and the like, that have been transected during surgery, and to achieve hemostasis or sutureless anastomosis.

As an alternative, this device can also be used as an adjunct to typical mechanical wound closure devices such as sutures, staples, and the like, where superior strength and sealing are required to prevent leaks of body fluids. Particular examples of this application include using the device as an adjunct for staple line or suture line in lung resection surgeries, blood vessel anastomosis, etc. The flexible substrate of the present invention can also be used as an adjunct to other methods of wound closure by other available sealants like biological formulations such as fibrin sealants, gelatin matrixes, or synthetic formulations like albumin-glutaraldehyde, polyethylenglycol hydrogel formulations, etc.

As an alternative, this device can be used as a patch to seal wounds or defects on organs such as, spleen, liver, ovaries, etc. or repair other areas in the body such as, the peritoneal wall following C-Sections.

The following examples are illustrative of the principles and practice of the present invention, although not limited thereto.

### EXAMPLES

### Example 1

### Evaluation of the Bonding Capabilities of an ORC Patch Coated with a Cyanoacrylate Adhesive

### Materials and Method

The ORC material chosen was an ORC woven fabric sold under the tradename INTERCEED (Lot# XMB686-3; EXP 2001-09). ORC strips (0.5″×1.5″) were cut from a 3″×4″ sheet then neutralized by immersion for 30 seconds in a bicarbonate solution (1% by weight) before being dried on paper towel.

The cyanoacrylate adhesive, a biodegradable cyanoacrylate monomer [3-(2-Cyano-acryloyloxy)-hexanoic acid ethyl ester], was synthesized according to the methods described in U.S. Pat. No. 7,238,828B2. Testing was performed on pig organs. A pig was anesthetized according to standard methods. A heating blanket was positioned on the animal's abdomen to keep the body temperature from dropping. As a routine procedure, the pig body temperature was measured via a rectal probe and found to be 97° F. The peritoneal cavity was opened and selected organs (peritoneal wall, small intestine, large intestine, spleen, and liver) were exposed.

Ten (10) microliters of the cyanoacrylate monomer were dispersed evenly over a 0.5″×0.5″ surface of each ORC strip. Each portion of the strip impregnated with the monomer was placed (by pressing for 5 seconds) on selected organs of the animal. Adhesiveness was then evaluated by pulling on the strip. Tests were performed on dry and wet surfaces. In one experiment, the neutralized ORC strip was wrapped around the intestine prior to the addition of the cyanoacrylate monomer.

Saline solution and gauze (tamponade) were used to simulate wet organ surfaces observed during surgical procedures as the surgeon performs rinses.

### Results

All tests demonstrated success in terms of adhesion of the ORC strips to the peritoneal cavity and to the selected organs (liver, spleen, large intestine, small intestine), either wet or dry. The method of applying the ORC strip and adhesive did not affect the ability of the cyanoacrylate to adhere the ORC to the site.

### Example 2

### Evaluation of the Wound Closure Capabilities of an ORC Patch Coated with a Cyanoacrylate Adhesive

### Materials and Method

The ORC material chosen was an ORC woven fabric sold under the tradename INTERCEED (Lot# XMB686-3; EXP 2001-09). ORC strips (0.5″×1.5″) were cut from a 3″×4″ sheet then neutralized by immersion for 30 seconds in a bicarbonate solution (1% by weight) before being dried on paper towel. The cyanoacrylate adhesive is a biodegradable cyanoacrylate monomer [3-(2-Cyano-acryloyloxy)-hexanoic acid ethyl ester] as described in Example 1.

### Ex-Vivo Testing

GI burst tests were performed on pig intestine immersed in water at 37° C. A 1 cm incision was performed longitudinally along a piece (approx 6″ long) of intestine and a suture point was placed in the middle of the incision. Four test groups were evaluated for wound closure ability: ORC was used “as is” as negative control; 100 microliters of the cyanoacrylate monomer were dispensed and spread along the approximated edges of the wound as positive control; ORC was neutralized then pat-dried on paper then covered with 100 microliters of sealant prior to its application to the dried wound; and ORC was neutralized then dried on paper then covered with 100 microliters of sealant prior to its application to the wound wetted with normal saline solution.

In all cases, 5 minutes curing time was allowed before performing a burst pressure test on the wound. The burst tests were performed by inflating the piece of intestine with air and monitoring the internal pressure until failure (rupture of the wound).

### Results

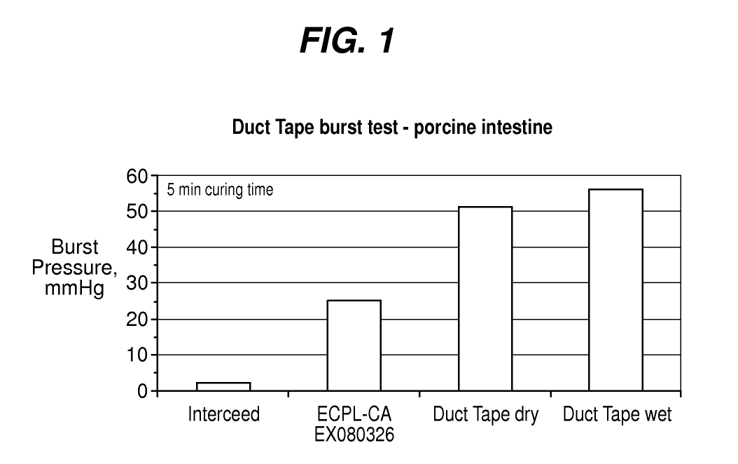

FIG. 1 shows the burst pressure required to rupture the wound for each of the 4 test groups. ORC alone had the lowest burst pressure as expected since ORC alone does not have substantial mechanical strength. The cyanoacrylate adhesive (positive control) sealed the wound up to 25 mmHg. Both ORC patches coated with the cyanoacrylate adhesive led to a significant improvement in terms of the increased resistance to burst (up to 55 mm Hg) of the repaired wound. The ORC patches coated with the cyanoacrylate adhesive performed equally well on a dried or wetted wound.

### Example 3

### Evaluation of the Wound Closure Capabilities of an ORC Patch Coated with a Cyanoacrylate Adhesive

### Materials and Method

The ORC material chosen was an ORC woven fabric sold under the tradename INTERCEED (Lot# XMB686-3; EXP 2001-09). ORC strips (0.5″×1.5″) were cut from a 3″×4″ sheet then neutralized by immersion for 30 seconds in a bicarbonate solution (1% by weight) before being dried on paper towel. The cyanoacrylate adhesive is a biodegradable cyanoacrylate monomer [3-(2-Cyano-acryloyloxy)-hexanoic acid ethyl ester] (ECPL-CA) as described in Example 1.

### Ex Vivo Testing

GI burst tests were performed on harvested pig intestine immersed in water at 37° C. A 1 cm incision was performed longitudinally along a piece (approx 6″ long) of intestine and a suture point was placed in the middle of the incision. Three test groups were evaluated for wound closure ability: 100 microliters of sealant ECPL-CA were dispensed and spread along the approximated edges of the wound and over a surface equal to one of ORC as positive control; ORC was neutralized, pat-dried on paper then applied to the dried wound and subsequently covered with 100 microliters of sealant; ORC was neutralized, lyophilized then applied to the dried wound and subsequently covered with 100 microliters of sealant.

In all cases, 5 minutes curing time was allowed before performing a burst pressure test on the wound. The burst tests were performed by inflating the piece of intestine with air and monitoring the internal pressure until failure (rupture of the wound).

### Results

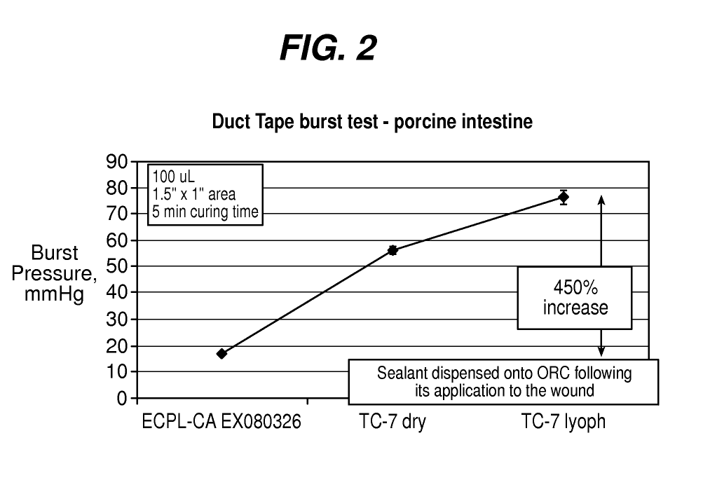

FIG. 2 shows the burst pressure for each of the three treatment groups. The cyanoacrylate adhesive (positive control) sealed the wound up to 18 mmHg. ORC patch paper dried post neutralization and coated with the cyanoacrylate adhesive led to a significant improvement in terms of the increased resistance to burst (57 mmHg) of the repaired wound (as seen before). ORC patch lyophilized post neutralization and coated with the cyanoacrylate adhesive led to a significant improvement (76 mm Hg) over the paper dried one in terms of the increased tensile strength of the repaired wound. Overall this represents a 450% improvement over the positive control in terms of the acute burst resistance of the wound. It also represents a 15% improvement over the partially neutralized non-lyophilized ORC.

### Example 4

### Partial Neutralization of ORC

For the purposes of this experiment an ORC woven fabric sold under the trade name SURGICEL was used. Different neutralization levels were obtained by immersing the ORC fabrics in 1% by weight sodium bicarbonate solution for selected periods of time ranging from 0 to 120 seconds. The pH of the ORC was measured as follows: 160+/−10 mg of woven fiber were placed into a glass vial to which 1 mL of distilled/deionized water was added. The mixture was shaken for 10 seconds and the pH was subsequently measured with a pH meter. Time to cyanoacrylate polymerization in the presence of the partially neutralized ORCs was measured as follows: pieces of ORC fabrics (0.25 in²) were placed onto agar gel plates, then 254 of Octyl Cyanoacrylate were dispensed on top of each piece. Time to polymerization was then evaluated visually as the time for the fabric to become rigid and as evidenced by color change from clear to opaque (see Table 1).

TABLE 1

| Time of immersion of ORC in | | Time to polymer- |
|---|---|---|
| sodium bicarbonate, seconds | pH | ization, minutes |
| 0 | 2.20 | 25 |
| 15 | 4.15 | 24 |
| 30 | 4.55 | 18 |
| 60 | 5.12 | 13 |
| 120 | 6.30 | 2 |

### Results

The level of neutralization of the ORC is required to be adapted for more or less reactive cyanoacrylates; however this experiment shows that a pH of from about 6 to about 7 is desired for polymerization of the octyl cyanoacrylate in the presence of the ORC.

Although this invention has been shown and described with respect to detailed embodiments thereof, it will be understood by those skilled in the art that various changes in form and detail thereof may be made without departing from the spirit and scope of the claimed invention.

We claim:

1. An oxidized regenerated cellulose adhesive tape, comprising:
a tape substrate comprising a dry, partially neutralized oxidized regenerated cellulose; and,
a bioabsorbable alpha-cyanoacrylate adhesive composition applied to the tape substrate.

2. The oxidized regenerated cellulose adhesive tape of claim 1 where the bioabsorbable alpha-cyanoacrylate adhesive composition comprises an alkyl ester alpha-cyanoacrylate monomer.

3. The oxidized regenerated cellulose adhesive tape of claim 2 wherein the alkyl ester alpha-cyanoacrylate monomer is selected from the group consisting of 3-(2-cyano-acryloyloxy)-butyric acid ethyl ester, 3-(2-cyano-acryloyloxy)-pentanoic acid ethyl ester, 3-(2-cyano-acryloyloxy)-hexanoic acid ethyl ester, 3-(2-cyano-acryloyloxy)-heptanoic acid ethyl ester, 3-(2-cyano-acryloyloxy)-octanoic acid ethyl ester, 4-(2-cyano-acryloyloxy)-hexanoic acid ethyl ester, 5-(2-cyano-acryloyloxy)-hexanoic acid ethyl ester, ethyl lactoyl cyanoacrylate, n-propyl lactoyl cyanoacrylate, isopropyl lactoyl cyanoacrylate, n-butyl lactoyl cyanoacrylate, isobutyl lactoyl cyanoacrylate, pentyl lactoyl cyanoacrylate, hexyl lactoyl cyanoacrylate, 2-ethyl-hexyl-lactoyl cyanoacrylate, n-octyl lactoyl cyanoacrylate, iso-octyl lactoyl cyanoacrylate, ethyl glycoloyl cyanoacrylate, n-propyl glycoloyl cyanoacrylate, isopropyl glycoloyl cyanoacrylate, n-butyl glycoloyl cyanoacrylate, isobutyl glycoloyl cyanoacrylate, pentyl glycoloyl cyanoacrylate, hexyl glycoloyl cyanoacrylate, 2-ethyl-hexyl-glycoloyl cyanoacrylate, n-octyl glycoloyl cyanoacrylate, and iso-octyl glycoloyl cyanoacrylate.

4. The oxidized regenerated cellulose adhesive tape of claim 3 wherein the alkyl ester alpha-cyanoacrylate monomer is selected from the group consisting of 3-(2-cyano-acryloyloxy)-butyric acid ethyl ester, 3-(2-cyano-acryloyloxy)-pentanoic acid ethyl ester, 3-(2-cyano-acryloyloxy)-hexanoic acid ethyl ester, 3-(2-cyano-acryloyloxy)-heptanoic acid ethyl ester, 3-(2-cyano-acryloyloxy)-octanoic acid ethyl ester, 4-(2-cyano-acryloyloxy)-hexanoic acid ethyl ester, and 5-(2-cyano-acryloyloxy)-hexanoic acid ethyl ester.

5. The oxidized regenerated cellulose adhesive tape of claim 4 wherein the alkyl ester alpha-cyanoacrylate monomer is 3-(2-cyano-acryloyloxy)-hexanoic acid ethyl ester.

6. The oxidized regenerated cellulose adhesive tape of claim 1 wherein the bioabsorbable alpha-cyanoacrylate adhesive composition comprises an alkyl ester alpha-cyanoacrylate monomer and at least one other alpha-cyanoacrylate monomer selected from the group consisting of an alkyl cyanoacrylate monomer and an alkoxyalkyl cyanoacrylate monomer.

7. The oxidized regenerated cellulose adhesive tape of claim 1, wherein the dry, partially neutralized oxidized regenerated cellulose is neutralized to a pH of from about 2 to about 9.

8. The oxidized regenerated cellulose adhesive tape of claim 5, wherein the dry, partially neutralized oxidized regenerated cellulose is neutralized to a pH of from about 5 to about 7.

9. The oxidized regenerated cellulose adhesive tape of claim 1, wherein the dry, tape substrate comprises a form selected from the group consisting of woven fabrics, nonwoven fabrics, foams, and particles.

10. The oxidized regenerated cellulose adhesive tape of claim 7, wherein the dry, partially neutralized oxidized regenerated cellulose is a woven fabric.

11. The oxidized regenerated cellulose adhesive tape of claim 7, wherein the dry, partially neutralized oxidized regenerated cellulose is a nonwoven fabric.

12. A method of making an oxidized regenerated cellulose adhesive tape, comprising the steps of:
providing a tape substrate comprising oxidized regenerated cellulose;
neutralizing the oxidized regenerated cellulose to a pH of from about 2 to about 9;
drying the neutralized oxidized regenerated cellulose;
providing an adhesive comprising an alpha-cyanoacrylate adhesive composition; and,
applying the alpha-cyanoacrylate adhesive composition to the tape substrate thereby producing an adhesive tape.

13. The method of claim 12 wherein the neutralizing of the oxidized regenerated cellulose is to a pH of from about 5 to about 7.

14. The method of claim 12, wherein the drying step comprises lyophilizing.

15. The method of claim 12, wherein the drying step comprises blotting.

16. The method of claim 12, additionally comprising the step of packaging the adhesive tape in a sterile package.

17. A method of treating tissue in a patient, comprising:
A. providing an oxidized regenerated cellulose adhesive tape, comprising: a tape substrate comprising dry, partially-neutralized oxidized regenerated cellulose; and,
B. a bioabsorbable alpha-cyanoacrylate adhesive composition applied to the tape substrate; and,
applying the adhesive tape to tissue in a patient.

18. The method of claim 17, wherein the tissue is approximated by the adhesive tape.

19. An oxidized regenerated cellulose adhesive tape kit, comprising:
a tape substrate comprising a dry, partially neutralized oxidized regenerated cellulose; and,
a bioabsorbable alpha-cyanoacrylate adhesive composition,
wherein the adhesive composition is applied to the tape substrate to form an oxidized regenerated cellulose adhesive tape.
